(19) United States
(12) Patent Application Publication
(10) Pub. No.: US 2007/0277331 A1
(43) Pub. Date: Dec. 6, 2007

(54) Protecting areas of skin proximate hair or nails from hair colors/ dyes and other contaminants

(75) Inventors: Jeffrey I. Goldstein, Cincinnati, OH (US); David R. Tubesing, Crestview Hills, KY (US)

Correspondence Address: JAGTIANI + GUTTAG, 10363-A DEMOCRACY LANE, FAIRFAX, VA, 22030, US

(73) Assignee: Ingenuity Products LLC, Cincinnati, OH (US)

(21) Appl. No.: 11/445,190

(22) Filed: Jun. 2, 2006

(51) Int. Cl. A61K 8/40; C11D 1/62

(52) U.S. Cl. 8/406; 424/70.28

(57) ABSTRACT

The present invention provides for selective and uniform delivery of compositions to protect certain targeted areas: (1) skin proximate colorable or dyeable hair from staining by hair colors or hair dyes; or (2) skin proximate nails against nail skin contaminants which may include hair color or hair dyes. Embodiments of products of the present invention may comprise the protective composition and a device for selective and uniform delivery of the composition to the targeted area of the skin. Products may also comprise a protective composition having a solid or semisolid consistency which is formed to enable selective and uniform delivery of the composition to the targeted area of the skin. Products may also comprise: (a) a hair color or hair dyeing kit comprising the protective composition and one or more hair color or hair dyeing components; or (b) a nail cosmetic kit comprising the protective composition and one or more nail cosmetic components.

### BACKGROUND

1. Field of the Invention

The present invention broadly relates to protecting areas of skin proximate colorable or dyeable hair on the head, eyebrows, etc., from staining by hair colors or hair dyes by selective and uniform delivery to such skin areas of a composition protective against staining by such hair colors or dyes. The present invention further broadly relates to protecting skin proximate nails from nail skin contaminants by selective and uniform delivery to such nail skin areas of a composition protective against hair colors, hair dyes, or other nail skin contaminants.

2. Related Art

When coloring or dyeing hair, even when the hair color or hair dye composition is applied with utmost care, coloring of the skin may not be avoidable. This coloring or staining of the skin may be unsightly and annoying on the visible parts of the skin directly proximate or adjoining the hairline, scalp, etc. Some dyes, such as some oxidation dyes, as well as some hair permanent waving solutions, may also cause discomfort, irritation, or sensitization of the scalp or adjoining skin, even if the contact time with the scalp/skin is of very short duration, and even if these oxidation dyes or hair waving solutions are rinsed away just after the dyeing operation.

So-called color-spot removers are known which may be employed after hair dyeing or color treatments are finished to remove spots of color from scalp, skin, adjoining hairline, etc. See Background of Invention of U.S. Pat. No. 5,500,218 (Kischka et al.), issued Mar. 19, 1996. However, subsequent removal of these spots of color may be irritating to the scalp or skin due to the cleaning materials contained in the color-spot remover compositions, as well as the mechanical rubbing which may be required to remove the spots of color. In addition, these color-spot removers may not be able to entirely eliminate the color from the stained skin. Even with the assistance of soap and scrubbing, removal of such stains may be difficult or impossible achieve.

In many methods of coloring hair, a peroxide, such as hydrogen peroxide, may be used to develop the color in oxidative dyes in situ. The reaction of this peroxide may not be readily controlled, and may act to increase the intensity of the staining reaction should excess peroxide be present on or in the hair. A hair or scalp treatment composition may be employed after or during the hair dyeing or coloring operation that contains an enzyme to arrest or retard this oxidative reaction that causes staining. See, for example, U.S. Pat. No. 6,497,892 (Pugliese et al.), issued Dec. 24, 2002; U.S. Pat. No. 4,129,644 (Kalopissis et al.), issued Dec. 12, 1978. Even if such an enzyme-containing composition is used to control the oxidative reaction, the staining and other adverse effects to the scalp, hairline and adjoining skin caused by the hair coloring or dyeing operation may have already occurred.

In addition to the scalp, hairline and adjoining skin, other portions of the body may be stained during this hair color and dyeing operation, including portions of the body of the person applying the hair dye or coloring product, such as a beautician. One part of the body that may be stained during hair color and dyeing operations is the cuticle area adjacent to the nail. Removing these stains from the cuticle area may be difficult to carry out and may be accompanied by some of the same or similar adverse effects that may occur during removal of such stains from the scalp, hairline and adjoining skin. In addition, the cuticle may be subjected to other types of staining, irritation, etc., during, for example, various cosmetic operations performed on fingernails and/or toenails. These nail cosmetic operations may include nail painting or coloring, nail repair, nail cleaning, nail polish removal, increasing the size of the nail (e.g., applying artificial nails), etc.

To protect the cuticle against such staining, irritation, etc., caused by such nail cosmetic operations, various types of cuticle shields, masks or guards have been suggested. See, for example, U.S. Pat. No. 6,085,756 (Sexton et al.), issued Jul. 11, 2000; U.S. Pat. No. 4,960,587 (Rucker), issued Oct. 2, 1990; U.S. Pat. No. 4,605,024 (Tremblay), issued Aug. 12, 1986; U.S. Pat. No. 2,220,363 (Austin et al.), issued Nov. 5, 1940. These cuticle shields or guards may or may not be easy to use, and may or may not provide adequate protection of the cuticle against staining, irritation, etc., caused by such nail cosmetic operations.

Accordingly, it would be desirable to provide: (1) protection of the scalp, hairline, or other areas of skin proximate hair on the head, eyebrows, etc., against staining from hair coloring or dyeing operations by selective and uniform delivery to such skin areas of a protective composition; and (2) protection of nail cuticles against staining, irritation, etc., by hair color and dyeing operations, nail cosmetic operations, or other cuticle contaminants by selective and uniform delivery to the cuticle of a protective composition.

### SUMMARY

According to a first broad aspect of the present invention, there is provided a method comprising the following steps:
(a) providing a composition which protects skin from staining by hair colors or hair dyes; and
(b) selectively and uniformly delivering the composition to skin proximate colorable or dyeable hair prior to subjecting the hair to hair colors or hair dyes to thereby protect the skin from staining by the hair colors or hair dyes.

According to a second broad aspect of the invention, there is provided a product comprising a hair color or hair dyeing kit comprising:
one or more hair color or hair dyeing components; and
a composition which protects skin from staining by hair colors or hair dyes.

According to a third broad aspect of the present invention, there is provided a method comprising the following steps:
(a) providing a composition which protects skin proximate a nail from nail skin contaminants; and
(b) selectively and uniformly delivering the composition to the skin prior to exposure to the nail skin contaminants to thereby protect the skin from the nail skin contaminants.

According to a fourth broad aspect of the invention, there is provided a product comprising a nail cosmetic kit comprising:
one or more nail cosmetic components; and
a composition which protects skin proximate a nail from nail skin contaminants.

According to a fifth broad aspect of the present invention, there is provided a product comprising:
a composition which protects skin from staining by hair colors or hair dyes or which protects skin proximate a nail from nail skin contaminants; and
a device for selective and uniform delivery of the composition to skin proximate colorable or dyeable hair or to skin proximate a nail to thereby protect the area of skin proximate the hair from staining by the hair colors or hair dyes, or to thereby protect the skin proximate the nail from nail skin contaminants.

According to a sixth broad aspect of the invention, there is provided a product comprising:
a composition which protects skin proximate colorable or dyeable hair from staining by hair colors or hair dyes, or which protects skin proximate a nail from nail skin contaminants;
wherein the composition has a solid or semisolid consistency and is formed to enable selective and uniform delivery of the composition to skin proximate colorable or dyeable hair, or to skin proximate a nail.

### BRIEF DESCRIPTION OF THE DRAWINGS

The invention will be described in conjunction with the accompanying drawings, in which:

FIG. 1 is a perspective view of an embodiment of a product according to the present invention comprising a device for selective and uniform delivery of a protective composition, with product cap removed to show dispensed product;

FIG. 2 is a sectional view of the embodiment of FIG. 1; and

FIG. 3 is a perspective view of another embodiment according to the present invention wherein the composition is dispensed in a chisel-shaped configuration.

### DETAILED DESCRIPTION

It is advantageous to define several terms before describing the invention. It should be appreciated that the following definitions are used throughout this application.

### Definitions

Where the definition of terms departs from the commonly used meaning of the term, applicant intends to utilize the definitions provided below, unless specifically indicated.

For the purposes of the present invention, the term “skin” refers to any epidermal tissue layer, membrane covering the human body epidermis including but not limited to scalp, hairline, areas on the face and around the ears and around the back of the neck, areas of skin proximate the nail, such as the nail cuticle, etc.

For the purposes of the present invention, the term “hair” refers to hair from the head or scalp, sideburns, nape of the neck, eyebrows, facial hair (e.g., mustache, beard, etc.) etc., that may be subjected to hair coloring or hair dyeing.

For the purposes of the present invention, the term “area of skin proximate hair” refers to skin that is adjacent, adjoining, near, close, neighboring, etc., hair, including but not limited to the scalp, hairline, ears, around the eyebrows, on the back of the neck (nape), chin, nose, lips, face, etc.

For the purposes of the present invention, the term “hair color” or “hair dye” refer interchangeably to one or more compositions, components, agents, etc., used to change, alter, restore, modify, etc., the color of human hair, either temporarily, semi-permanently or permanently.

For the purposes of the present invention, the term “nail” refers to the keratinous sheath on the upper end of each finger or toe and includes the terms fingernail and toenail.

For the purposes of the present invention, the term “skin proximate the nail” refers to skin adjacent, adjoining, surrounding, etc., any portion of the nail, including the nail cuticle, finger tips, skin along the sides and beneath the nail, etc.

For the purposes of the present invention, the term “nail cuticle” refers to the upper portion of skin adjacent or adjoining the nail.

For the purposes of the present invention, the term “nail skin contaminant” refers to any material, composition, etc., that may stain, irritate or cause swelling to (e.g., cause an allergic reaction to), discolor, inflame, cause itching to, etc., skin proximate the nail, such as, for example, the a nail cuticle, including but not limited to hair colors, hair dyes, hair permanent waving solutions, nail polish and other nail cosmetic agents, paint and other coatings, grease, dirt, fiberglass, other dyes, inks, pigments, solvents, other chemicals (e.g., petrochemicals), etc.

For the purposes of the present invention, the term “staining” refers to any undesired, unwanted, etc., coloring or dyeing of skin.

For the purposes of the present invention, the term “protects skin from staining by hair colors or hair dyes” refers to the ability to minimize, diminish, reduce, lessen, decrease, avoid, prevent, inhibit, eliminate, etc., staining of the skin by hair colors or hair dyes.

For the purposes of the present invention, the term “protects skin proximate a nail from nail skin contaminants” refers to the ability to minimize, diminish, reduce, lessen, decrease, avoid, prevent, inhibit, eliminate, etc., the presence of such contaminants on skin proximate the nail, such as, for example, the nail cuticle.

For the purposes of the present invention, the terms “selective and uniform delivery,” “selectively and uniformly delivering” and similar terms refer to the ability to deliver the composition to a relatively specific, targeted, etc., area of the skin and to provide relatively consistent delivery of the composition in terms of one or more of the length, width and thickness (depth) of the delivered composition. For example, compositions that are selectively delivered provide a precise, targeted quantity of composition to the area of skin to be protected, such as the forehead in close proximity to the hairline, around the eyebrows, etc., without coating the adjacent hair. For example compositions which are uniformly delivered provide a relatively uniform thickness (e.g., do not have, for example, peaks and valleys in terms of the thickness (depth) of the composition delivered), provide a relatively uniform width (e.g., do not have a varying width, for example, a decreasing or narrowing width at the end of the delivery of the composition), provide a relatively consistent volume or amount, etc., of the delivered composition each time the composition is deposited, applied, etc., to the targeted area of skin.

For the purposes of the present invention, the term “to provide a consistent thickness of the delivered composition” means a thickness (depth) which does not substantially vary (e.g., no peaks and valleys) over the area the composition is delivered to.

For the purposes of the present invention, the term “to provide a consistent width of the delivered composition” means a width that does not vary substantially (e.g., no narrowing or widening) over the area the composition is delivered to. For example, embodiments of the present invention may deliver the composition as a parallel strip which is within about ⅛ inches or less, more typically about 1/16 inches or less, of the targeted area.

For the purposes of the present invention, the term “device for selective and uniform delivery” refers to any device that may used to selectively and uniformly deliver compositions, e.g., delivers a consistent thickness, width, volume, amount, etc., of the delivered composition. The device may be associated with, packaged with, contain, etc., the composition to be selectively and uniformly delivered or may be separate from the composition prior to selective and uniform delivery. The device may also be provided with a component for shaping, forming, molding, casting, contouring, etc., the composition prior to or during selective and uniform delivery, such as an orifice, nozzle, guide, funnel, etc. Selective and uniform delivery devices may include but are not limited to applicators, dispensers, containers, etc., such as roll-ons, tubes (e.g., squeeze tubes), tubules, pacquettes, swabs, pads, brushes, syringe, syringe with applicator, wipes, sticks, etc. See, for example, U.S. Pat. No. 5,733,058 (Hofmann), issued Mar. 31, 1998, the disclosure of which is herein incorporated by reference, for an illustrative dispenser container which may be used in solid or semi-solid embodiments of products according to the present invention.

### Description

The present invention provides for selective and uniform delivery of compositions to protect certain targeted areas: (1) skin proximate hair from staining by hair colors or hair dyes; or (2) skin proximate nails against nail skin contaminants which may include hair color or hair dyes. The delivered composition provides a selective and targeted application having a consistent thickness and width of the delivered composition. Embodiments of products of the present invention may comprise the protective composition and a device for selective and uniform delivery of the composition to the targeted area. Embodiments of products of the present invention may also comprise a protective composition having a solid or semisolid consistency which is formed to enable selective and uniform delivery of the composition to the targeted area. Embodiments of products of the present invention may also comprise: (a) a hair color or hair dyeing kit comprising the protective composition and one or more hair color or hair dyeing components; or (b) a nail cosmetic kit comprising the protective composition and one or more nail cosmetic components.

Compositions containing enzymes that inactivate the peroxides in hair coloring compositions for protecting the skin have been suggested. See U.S. Pat. No. 4,129,644 (Kalopissis et al.); U.S. Pat. No. 6,309,656 (Pugliese et al); U.S. Pat No. 6,497,892 (Pugliese et al); and U.S. Pat. Application No. 2002/0044916 (Pugliese et al). No mention is made of selectively or uniformly applying these enzyme-containing to the area to be treated with the composition. U.S. Pat. No. 5,500,218 (Kischka et al), issued Mar. 19, 1996, describes applying a skin protective composition to skin adjoining the hairline before hair dyeing to prevent skin coloring. This skin protective composition is said to be “uniformly applied to the skin adjoining the hairline, normally in a width of 1 to 2 cm, possibly with the use of a spatula, and the hair is then treated with hair dye.” See col. 2, lines 58-63 and Example 5. No mention is made of the skin protective composition being applied to provide a consistent thickness of the delivered composition, or that the composition is delivered by a device that provides a consistent thickness of the delivered composition. It has also been found that use of a spatula, especially on the curvaceous areas of the skin, may be unable or incapable of delivering a skin protective composition to a targeted area of the skin (for example, to the forehead in close proximity to the hairline without coating the hair itself), or with a consistent uniformity in thickness (depth), width or volume. As the skin protective composition is delivered by the spatula to a curvaceous skin area, it has been found that peaks and valleys are created in the thickness of the delivered composition, with the width of the delivered composition becoming progressively narrower at the end of the delivery from the spatula. A spatula may also be unable to deliver a consistent volume, amount, etc., of the composition from the container (e.g., tub or pot) to the targeted area which may also prevent uniform delivery of the composition.

To protect skin proximate nails, such as nail cuticles against staining, irritation, etc., caused by nail cosmetic operations, various types of cuticle shields, masks or guards have been suggested. See, for example, U.S. Pat. No. 6,085,756 (Sexton et al.), issued Jul. 11, 2000; U.S. Pat. No. 4,960,587 (Rucker), issued Oct. 2, 1990; U.S. Pat. No. 4,605,024 (Tremblay), issued Aug. 12, 1986; U.S. Pat. No. 2,220,363 (Austin et al.), issued Nov. 5, 1940. The use of compositions that may be temporary or removable for protecting skin proximate nails, such as nail cuticles, against such contaminants are not mentioned, nor is selective and uniformly delivery of such compositions to skin proximate nails, such as the nail cuticle, taught.

Embodiments of the protective compositions according to the present may have a solid, semi-solid, liquid or thixotropic consistency. Protective compositions having solid or semi-solid consistencies may provide advantages in forming the composition into a shape prior to selective and uniform delivery thereof. The protective composition may be in the form of a solution, suspension, gel, stick, cream, balm, lotion, ointment, emulsion, etc. The protective composition and/or the dispenser for the composition may be formulated or shaped to provide, for example, a chisel-shaped point for ease of selective and uniform delivery, may have, for example, an oval-shaped cross-section (e.g., is provided in an oval-shaped dispenser) to enable wide or narrow applications of the composition as the targeted area requires, to provide an ergonomically friendly application of the composition, etc.

Embodiments of the protective compositions of the present invention may comprise one or more of the following emollients: petrolatum (e.g., liquid or semi-solid petrolatum), lanolin, spermacetti, saturated and unsaturated C₁₄-C₂₃ alcohols and mixtures thereof such as myristyl alcohol, palmityl alcohol, stearyl alcohol, cetyl alcohol, etc., cetyl ester wax, glyceryl esters of saturated and unsaturated C₁₄-C₂₃ acids, saturated and unsaturated fatty acids such as C₁₄ to C₂₃ acids and their oligomers having 2 to 5 units, esters of saturated and unsaturated fatty acids such as C₁₄ to C₂₃ acids such as isopropyl myristate, isopropyl palmitate, isopropyl isostearate, isostearyl isostearate, diisopropyl sebacate, propylene glycol dipelargonate, 2-ethylhexyl isononoate, 2-ethylhexyl stearate, C₁₂-C₁₆ fatty alcohol lactate, isopropyl lanolate, 2-ethyl-hexyl salicylate and mixtures thereof, various solid or semi-solid waxes (e.g., candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), paraffin waxes, microwaxes; chemically modified waxes (hard waxes) such as, for example, montan ester waxes, sasol waxes, hydrogenated jojoba waxes, synthetic waxes such as, for example, polyalkylene waxes, polyethylene glycol waxes, and silicone waxes, etc.), liquid silicones such as, for example, dimethylpolysiloxane, (e.g., dimethicone, a dimethyl polysiloxane endblocked with trimethyl units), diethylpolysiloxane, dimethylpolysiloxane-diphenylpolysiloxane, cyclomethicone, trimethylpolysiloxane, diphenylpolysiloxane, and mixtures thereof, triglyceride oils (e.g., purified hydrogenated or non-hydrogenated vegetable oils such as canola oil, soybean oil, corn oil, wheat germ oil, rice bran oil, and the like or mixtures thereof), clays, shea butter, mango butter, glycerin, etc. The protective composition may include other skin care components such as emulsifiers, surfactants, water, etc.

To improve the ability to repel hair color or hair dyes, the protective composition may comprise hair color/hair dye repellant cationic materials such as quaternary ammonium compounds (e.g., quaternary ammonium chlorides, bromides, or iodides) having, for example, one or two long chain (e.g., C₁₄-C₂₂)alkyl (e.g., substantially linear alkyl, alkylamidoalkyl, etc) groups such as myristyl, palmityl, stearyl, arachidyl, behenyl etc., or a mixture thereof, and two or three shorter chain (e.g., C₁-C₄) such as methyl, ethyl, propyl, isopropyl, etc., or a mixture thereof, as well as other cationic materials having similar functional properties. Suitable cationic materials for use herein behenamidopropyl dimethylamine behenate, behentrimethylammonium chloride, etc. Other optional components may include preservatives, antimicrobials, antioxidants, fragrances, moisturizers, aloe, vitamins, minerals, herb, proteins, sunscreens, exfoliates, etc. Because the skin is negatively charged, protective compositions containing these cationic materials (which are positively charged), when applied to the skin, attach thereto by mechanical, electrical and/or ionic attraction. By contrast, the cationic materials in the protective compositions repel positively charged components, such as, for example, those present in hair dyes. In addition, the cationic materials in the protective composition may neutralize many sites on the skin that are negatively charged. As a result, these negatively charged sites on the skin are unavailable to attract the positively charged components, such as those present in hair dyes.

Selective and uniformly delivery of the composition, as well as delivery of the composition in a consistent thickness, may be carried out by selectively and uniformly, spreading, squeezing, distributing, dispensing, applying, wiping, rolling, coating, painting, spraying, pouring, etc., to the targeted area of skin. Typically, selective and uniform delivery is achieved by dispensing, applying, rolling, coating, painting, etc., the targeted area of skin.

The protective composition may be included as a component in a hair coloring or hair dyeing kit or as a components of a nail cosmetic kit. In addition to the protective composition, the hair coloring or hair dyeing kit may further include hair coloring and/or hair dyeing components, one or more of: combs, curlers, sponges, gloves, etc., and combinations thereof. In addition to the protective composition, the nail cosmetic kit may further include one or more of nail coloring components, nail polish components, artificial nails, nail jewelry or decorations, files, Emory boards, nail scissors, cutters, and trimmers, etc., and combinations thereof.

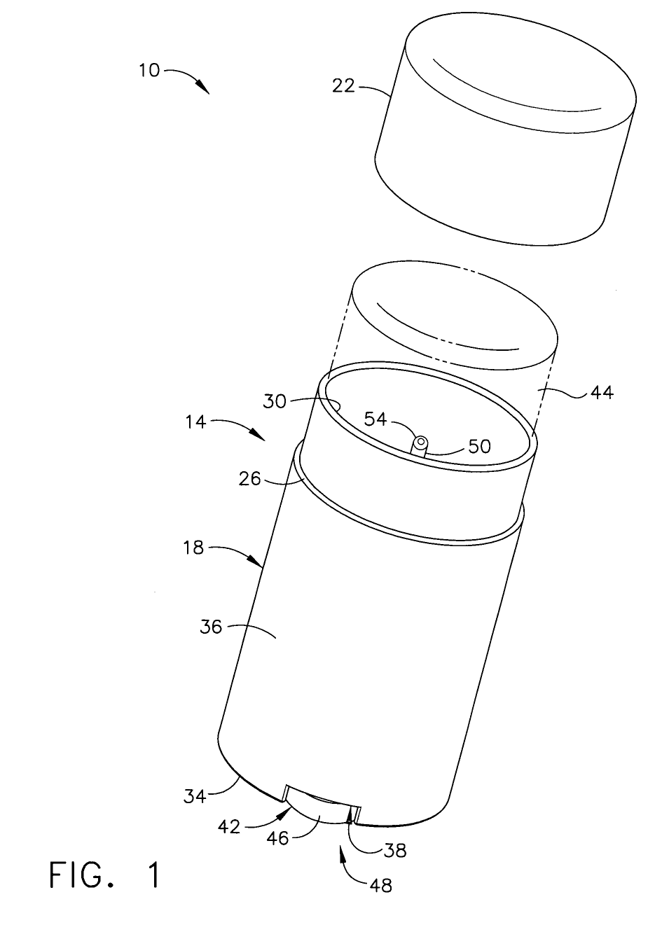

An embodiment of a product of the present invention comprising a device for selective and uniform delivery of a protective composition in the form of a stick having a solid or semi-solid consistency and an oval-shaped cross-section is shown FIG. 1 and is referred to generally as 10. Referring to FIG. 1, product 10 comprises a dispenser 14 comprising a housing 18 having, for example, a generally oval-shaped cross-section and a cap 22 having a corresponding generally oval-shaped cross-section. Housing 18 has an upper composition dispensing and cap receiving end 22 also having a corresponding generally oval-shaped cross-section and is provided with a corresponding generally oval-shaped opening 30 through which product may be dispensed. Housing 18 further includes a lower end 34 and a main body portion 36 having a generally oval-shaped cross-section connecting upper end 22 and lower end 34. Lower end 34 is provided with an annular recessed area, indicated as 38.

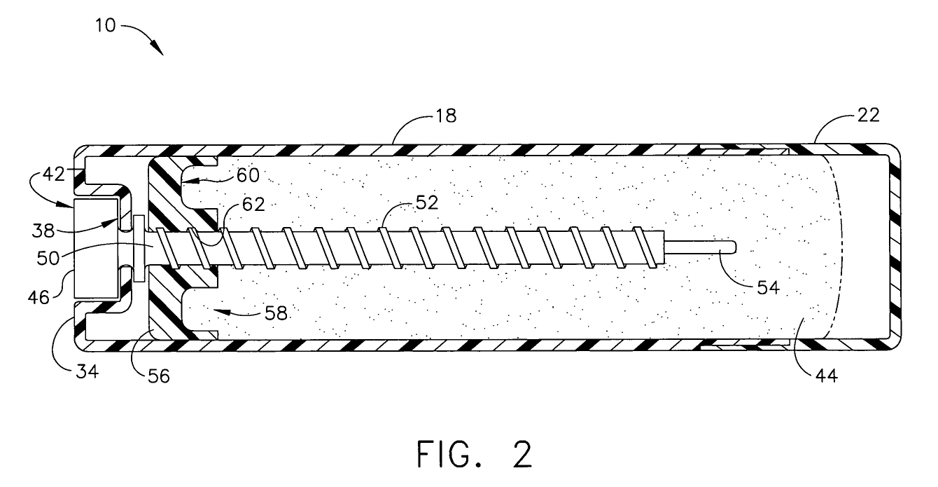

Referring to FIGS. 1-2, dispenser 14 further comprises a composition advancing and retrieving mechanism 42 which causes the solid or semi-solid protective composition, indicated generally as 44, to be dispensed through opening 30 or to be retrieved within housing 18. Mechanism 42 comprises actuating knob in the form of finger wheel 46 positioned within recessed area 38 in the bottom end of housing 18. Area 38 is provided with openings 48 so that fingerwheel 46 may be rotated or twisted to dispense composition 44 from or retrieve composition 44 within housing 18.

Mechanism 42 further comprises an elongated shaft or stem 50 which is connected a one end to fingerwheel 46. As shown in FIG. 2, stem 50 is inserted, through recessed area 38 of bottom end 34 of housing 18 and rotates in response to the twisting or rotation of fingerwheel 46. Stem 50 is provided with a threaded portion indicated as 52, the upper section of which indicated as 54, may impale or be surrounded by the protective composition 44. A generally cup-shaped follower 56 is also provided which has a recess 58 for receiving and engaging composition 44 and a bottom surface 60 which assists in advancing composition 44 when fingerwheel 46 is rotated or twisted in the appropriate direction.

Follower 56 is provided with a threaded aperture 62 in the center of bottom surface through which the threaded portion 52 of stem 50 is inserted so that stem 50 may move follower 56 upwardly or downwardly within housing 18 in response to the rotation or twisting of fingerwheel 46. Depending on which direction fingerwheel 46 is rotated or twisted, follower 56 may move upwardly along threaded portion 52 of stem 50 to dispense composition 44 from housing 18, or may move downwardly along threaded portion 52 of stem 50 to retrieve composition within housing 18, in response to the needs of the user.

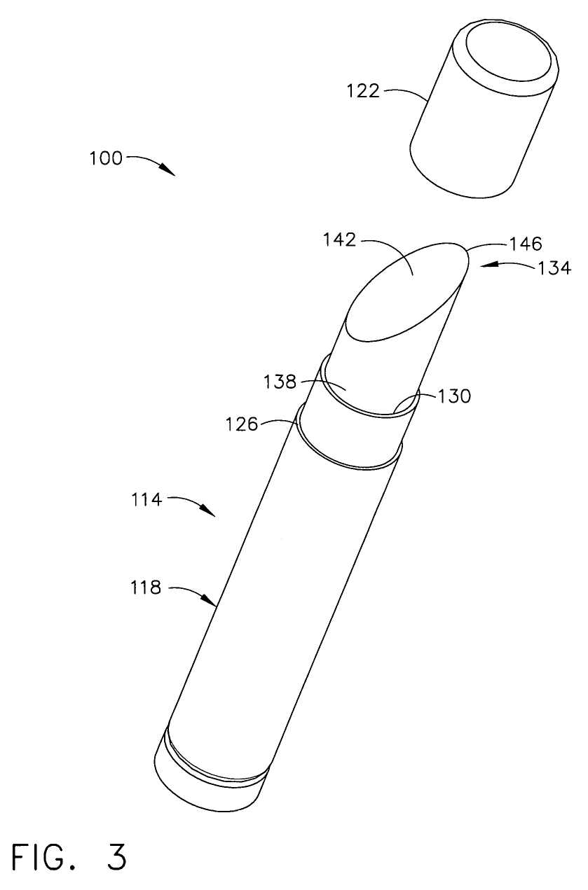

Another embodiment of the present invention for selective and uniform delivery of a protective composition having a solid or semi-solid consistency in a different shaped form is shown FIG. 3 and is referred to generally as 100. Product 100 may comprise a dispenser 114 comprising a housing 118 having, for example, a generally circular-shaped cross-section, and a cap, indicated generally as 122, having a corresponding generally circular-shaped cross-section. Housing 118 has an upper composition dispensing and cap receiving end 126 also having a corresponding generally circular-shaped cross-section and is provided with a corresponding generally circular-shaped opening 130 thorough which product may be dispensed. As shown in FIG. 3, the dispensed product, indicated generally as 134, has what is referred to as a chisel-shaped configuration with a generally cylindrical lower main portion, indicated generally as 138, and a slanted upper surface, indicated as 142, having a generally elongated oval-shaped appearance. Upper surface 142 provides a relatively sharply defined upper product application point, indicated as 146, which enables the user to apply the product relatively thinly, selectively and uniformly to the targeted area. Like product 10 shown in FIGS. 1-2, the product 100 shown in FIG. 3 may be provided with a similar or identical mechanism as 42 of product 10 for advancing or retrieving product 134 from or within housing 118.

### EXAMPLES

### Example I

The following is an example a composition for protecting skin against staining by hair colors or hair dyes, or other nail skin contaminants:

| Ingredient | Amount (Weight %) |
|---|---|
| Petrolatum USP Snow White | 45–50 |
| Cetyl Alcohol (optional) | 8–12 |
| Stearyl Alcohol & Behenamidopropyl | 3–8 |
| Dimethylamine Behenate | |
| Silicone Wax (optional) | 3–8 |
| Vegetable or Mineral Oil (optional) | 22–28 |
| Cyclomethicone | 1–5 |
| Mango Butter | 3–8 |

The above ingredients are mixed together in the order indicated above. Other than the cyclomethicone, the ingredients are mixed at a temperature in the range of from about 70 to about 85° C. The cyclomethicone is added in at a temperature of less than about 60° C.

### Example II

The following is another example a composition for protecting skin against staining by hair colors or hair dyes, or other nail skin contaminants:

| Ingredient | Amount (Weight %) |
|---|---|
| Petrolatum USP Snow White | 45–50 |
| Cetyl Alcohol (optional) | 8–12 |
| Stearyl Alcohol & Behentrimethylammonium | 3–8 |
| chloride | |
| Silicone Wax (optional) | 3–8 |
| Vegetable or Mineral Oil (optional) | 22–28 |
| Cyclomethicone | 1–5 |
| Mango Butter | 3–8 |

The above ingredients are mixed together in the order indicated above. Other than the cyclomethicone, the ingredients are mixed at a temperature in the range of from about 70 to about 85° C. The cyclomethicone is added in at a temperature of less than about 60° C.

All documents, patents, journal articles and other materials cited in the present application are hereby incorporated by reference.

Although the present invention has been fully described in conjunction with several embodiments thereof with reference to the accompanying drawings, it is to be understood that various changes and modifications may be apparent to those skilled in the art. Such changes and modifications are to be understood as included within the scope of the present invention as defined by the appended claims, unless they depart therefrom.

What is claimed is:

1. A method comprising the following steps:
(a) providing a composition which protects skin from staining by hair colors or hair dyes; and
(b) selectively and uniformly delivering the composition to skin proximate colorable or dyeable hair prior to subjecting the hair to hair colors or hair dyes to thereby protect the skin from staining by the hair colors or hair dyes.

2. The method of claim 1, wherein step (b) is carried out by delivering the composition to skin proximate the head or scalp.

3. The method of claim 1, wherein the composition of step (a) comprises an emollient.

4. The method of claim 3, wherein the composition of step (a) comprises one or more of petrolatum, a saturated and unsaturated C₁₄-C₂₃ alcohol, silicone wax, or liquid silicone.

5. The method of claim 3, wherein the composition of step (a) comprises a hair color/hair dye repellant cationic material.

6. The method of claim 5, wherein the composition of step (a) comprises one or more of behenamidopropyl dimethylamine behenate, behentrimethylammonium chloride.

7. The method of claim 1, wherein step (b) is carried out by selectively and uniformly dispensing, applying, rolling, coating or painting the composition on the skin.

8. The method of claim 7, wherein step (b) is carried out to provide a consistent uniformity in thickness and width of delivered composition.

9. The method of claim 7, wherein the composition provided in step (a) has a solid or semi-solid consistency.

10. A product comprising a hair color or hair dyeing kit comprising:
one or more hair color or hair dyeing components; and
a composition which protects skin from staining by hair colors or hair dyes.

11. The product of claim 10, wherein the kit comprises one or more of: combs, curlers, sponges, or gloves.

12. The product of claim 10, wherein the composition is in the form of a solution, suspension, gel, stick, cream, balm, lotion, ointment, or emulsion.

13. The product of claim 12, wherein the composition is shaped.

14. The product of claim 12, which comprises a dispenser for the composition and wherein the composition is dispensed from the dispenser in a shaped configuration or cross-section.

15. The product of claim 13, wherein the composition is dispensed in a chisel-shaped configuration or an oval-shaped cross-section.

16. The product of claim 10, wherein the composition comprises an emollient.

17. The product of claim 16, wherein the composition or more of petrolatum, a saturated and unsaturated C₁₄-C₂₃ alcohol, silicone wax, or liquid silicone.

18. The product of claim 17, wherein the composition comprises a hair color/hair dye repellant cationic material.

19. The product of claim 18, wherein the composition comprises one or more of behenamidopropyl dimethylamine behenate or behentrimethylammonium chloride.

20. A method comprising the following steps:
(a) providing a composition which protects skin proximate a nail from nail skin contaminants; and
(b) selectively and uniformly delivering the composition to the skin prior to exposure to the nail skin contaminants to thereby protect the skin from the nail skin contaminants.

21. The method of claim 20, wherein step (b) is carried out by delivering the composition to the nail cuticle.

22. The method of claim 21, wherein the composition of step (a) comprises an emollient.

23. The method of claim 21, wherein the composition of step (a) comprises one or more of petrolatum, a saturated and unsaturated C₁₄-C₂₃ alcohol, silicone wax, or liquid silicone.

24. The method of claim 22, wherein the composition of step (a) comprises a hair color/hair dye repellant cationic material.

25. The method of claim 24, wherein the composition of step (a) comprises one or more of behenamidopropyl dimethylamine behenate or behentrimethylammonium chloride.

26. The method of claim 21, wherein step (b) is carried out by selectively and uniformly dispensing, applying, rolling, coating or painting the composition on the nail cuticle.

27. The method of claim 26, wherein step (b) is carried out to provide a consistent uniformity in thickness and width of delivered composition.

28. The method of claim 26, wherein the composition of step (a) has a solid or semi-solid consistency.

29. A product comprising a nail cosmetic kit comprising:
one or more nail cosmetic components; and
a composition which protects skin proximate a nail from nail skin contaminants.

30. The product of claim 29, wherein the kit comprises one or more of: nail coloring components, nail polish components, artificial nails, nail jewelry or decorations, files, Emory boards, nail scissors, cutters, and trimmers.

31. The product of claim 29, wherein the composition is in the form of a solution, suspension, gel, stick, cream, balm, lotion, ointment, or emulsion.

32. The product of claim 31, wherein the composition is shaped.

33. The product of claim 32, which comprises a dispenser for the composition and wherein the composition is shaped by the dispenser.

34. The product of claim 33, wherein the composition is dispensed in a chisel-shaped configuration or an oval-shaped cross-section.

35. The product of claim 29, wherein the composition comprises an emollient.

36. The product of claim 35, wherein the composition comprises one or more of petrolatum, a saturated and unsaturated C₁₄-C₂₃ alcohol, silicone wax, or liquid silicone.

37. The product of claim 35, wherein the composition comprises a hair color/hair dye repellant cationic material.

38. The product of claim 37, wherein the composition comprises one or more of behenamidopropyl dimethylamine behenate or behentrimethylammonium.

39. A product comprising:
a composition which protects skin from staining by hair colors or hair dyes or which protects skin proximate a nail from nail skin contaminants; and
a device for selective and uniform delivery of the composition to skin proximate colorable or dyeable hair or to skin proximate a nail to thereby protect the area of skin proximate the hair from staining by the hair colors or hair dyes, or to thereby protect the skin proximate the nail from nail skin contaminants.

40. The product of claim 39, wherein the composition is formulated to protect the skin from staining by hair colors or hair dyes.

41. The product of claim 39, wherein the composition is formulated to protect the skin proximate the nail from nail skin contaminants.

42. The product of claim 39, wherein the composition is in the form of a solution, suspension, gel, stick, cream, balm, lotion, ointment, or emulsion.

43. The product of claim 42, wherein the composition is shaped by the dispenser.

44. The product of claim 43, wherein the composition has a solid or semi-solid consistency.

45. A product comprising:
a composition which protects skin proximate colorable or dyeable hair from staining by hair colors or hair dyes, or which protects skin proximate a nail from nail skin contaminants;
wherein the composition has a solid or semisolid consistency and is formed to enable selective and uniform delivery of the composition to skin proximate colorable or dyeable hair, or to skin proximate a nail.

46. The product of claim 45, wherein the composition is formulated to protect the skin from staining by hair colors or hair dyes.

47. The product of claim 45, wherein the composition is formulated to protect the skin proximate the nail from nail skin contaminants.

48. The product of claim 45, wherein the composition is dispensed in a chisel-shaped configuration or an oval-shaped cross-section.
